(19) United States
(12) Patent Application Publication
(10) Pub. No.: US 2008/0319522 A1
(43) Pub. Date: Dec. 25, 2008

(54) ANEURYSM FILLER DETACHER

(76) Inventors: Cathleen von Lehe, Rogers, MN (US); Marie F. Calabria, Plymouth, MN (US); Jeffrey A. Lee, Plymouth, MN (US)

Correspondence Address: SCHWEGMAN, LUNDBERG & WOESSNER, P.A., P.O. BOX 2938, MINNEAPOLIS, MN, 55402, US

(21) Appl. No.: 11/767,290

(22) Filed: Jun. 22, 2007

(51) Int. Cl. A61F 2/06

(52) U.S. Cl. 623/1.11

(57) ABSTRACT

An aneurysm filler detacher mechanism comprising a proximal push support comprising a copper or silver core wire, an insulation layer positioned around the core wire, and an annular flexible layer positioned around the insulation layer.

### FIELD

The inventive subject matter described herein relates to an aneurysm filler detacher mechanism and to a method for repairing an aneurysm. The inventive subject matter also relates to a method for making, a method for using and to a method for detaching an aneurysm filler detacher mechanism.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the products, processes and data as described below and in the tables that form a part of this document: Copyright 2007, Neurovasx, Inc. All Rights Reserved.

### BACKGROUND OF THE INVENTION

An aneurysm is a balloon-like swelling in a wall of a blood vessel. Aneurysms result in weakness of the vessel wall in which it occurs. This weakness predisposes the vessel to tear or rupture with potentially catastrophic consequences for any individual having the aneurysm. Vascular aneurysms are a result of an abnormal dilation of a blood vessel, usually resulting from disease and/or genetic predisposition which can weaken the arterial wall and allow it to expand. Aneurysm sites tend to be areas of mechanical stress concentration so that fluid flow seems to be the most likely initiating cause for the formation of these aneurysms.

Aneurysms in cerebral circulation tend to occur in an anterior communicating artery, posterior communicating artery, and a middle cerebral artery. The majority of these aneurysms arise from either curvature in the vessels or at bifurcations of these vessels. The majority of cerebral aneurysms occur in women. Cerebral aneurysms are most often diagnosed by the rupture and subarachnoid bleeding of the aneurysm.

Cerebral aneurysms are most commonly treated in open surgical procedures where the diseased vessel segment is clipped across the base of the aneurysm. While considered to be an effective surgical technique, particularly considering an alternative which may be a ruptured or re-bleed of a cerebral aneurysm, conventional neurosurgery suffers from a number of disadvantages. The surgical procedure is complex and requires experienced surgeons and well-equipped surgical facilities. Surgical cerebral aneurysm repair has a relatively high mortality and morbidity rate of about 2% to 10%.

Current treatment options for cerebral aneurysm fall into two categories, surgical and interventional. The surgical option has been the long held standard of care for the treatment of aneurysms. Surgical treatment involves a long, delicate operative procedure that has a significant risk and a long period of postoperative rehabilitation and critical care. Successful surgery allows for an endothelial cell to endothelial cell closure of the aneurysm and therefore a cure for the disease. If an aneurysm is present within an artery in the brain and bursts, this creates a subarachnoid hemorrhage, and a possibility that death may occur. Additionally, even with successful surgery, recovery takes several weeks and often requires a lengthy hospital stay.

In order to overcome some of these drawbacks, interventional methods and prostheses have been developed to provide an artificial structural support to the vessel region impacted by the aneurysm. The structural support must have an ability to maintain its integrity under blood pressure conditions and impact pressure within an aneurysmal sac and thus prevent or minimize a chance of rupture. U.S. Pat. No. 5,405,379 to Lane, discloses a self-expanding cylindrical tube which is intended to span an aneurysm and result in isolating the aneurysm from blood flow. While this type of stent-like device may reduce the risk of aneurysm rupture, the device does not promote healing within the aneurysm. Furthermore, the stent may increase a risk of thrombosis and embolism. Additionally, the wall thickness of the stent may undesirably reduce the fluid flow rate in a blood vessel. Stents typically are not used to treat aneurysms in a bend in an artery or in tortuous vessels such as in the brain because stents tend to straighten the vessel.

U.S. Pat. No. 5,354,295 to Guglielmi et al., describes a type of vasoclusion coil. Disadvantages of use of this type of coil are that the coil may compact, may migrate over time, and the coil does not optimize the patient's natural healing processes.

### IN THE FIGURES

FIG. 1 is a cross-sectional view of a proximal push support embodiment for an aneurysm filler detacher mechanism.

FIG. 2 is a cross-sectional view of another proximal push support embodiment for an aneurysm filler detacher mechanism.

FIG. 3 is a cross-sectional view of another proximal push support embodiment for an aneurysm filler detacher mechanism.

FIG. 4 is a cross-sectional view of another proximal push support embodiment for an aneurysm filler detacher mechanism.

FIG. 5 is a cross-sectional view of another proximal push support embodiment for an aneurysm filler detacher mechanism.

FIG. 6 is a cross-sectional view of another proximal push support embodiment for an aneurysm filler detacher mechanism.

FIG. 7 is a cross-sectional view of another proximal push support embodiment for an aneurysm filler detacher mechanism.

FIG. 8 is a cross-sectional view of another proximal push support embodiment for an aneurysm filler detacher mechanism.

FIG. 9 is a cross-sectional view of a radiopaque tip/coil section embodiment for an aneurysm filler detacher mechanism.

FIG. 10 is a cross-sectional view of another radiopaque tip/coil section embodiment for an aneurysm filler detacher mechanism.

FIG. 11 is a cross-sectional view of another proximal push support embodiment for an aneurysm filler detacher mechanism.

### DESCRIPTION

Although detailed embodiments of the invention are disclosed herein, it is to be understood that the disclosed embodiments are merely exemplary of the invention that may be embodied in various and alternative forms. Specific structural and functional details disclosed herein are not to be interpreted as limiting, but merely as a basis for teaching one skilled in the art to variously employ the aneurysm filler detacher mechanism embodiments. Throughout the drawings, like elements are given like numerals.

Referred to herein are trade names for materials including, but not limited to, polymers and optional components. The inventors herein do not intend to be limited by materials described and referenced by a certain trade name. Equivalent materials (e.g., those obtained from a different source under a different name or catalog (reference) number to those referenced by trade name may be substituted and utilized in the methods described and claimed herein. All percentages and ratios are calculated by weight unless otherwise indicated. All percentages are calculated based on the total composition unless otherwise indicated. All component or composition levels are in reference to the active level of that component or composition, and are exclusive of impurities, for example, residual solvents or by-products, which may be present in commercially available sources.

Embodiments of the delivery detacher mechanism described herein include a device that functions as both a delivery mechanism and a detachment mechanism for a catheter and includes a main body having distal flexibility associated with a delivery mechanism portion, a proximal push support, a detachment mechanism that is concentrated, and a radiopaque tip/coil section. Delivery detacher mechanism embodiments described herein have substantially the same level of flexibility and push support at the detacher portion as is at the distal end as a delivery mechanism. Embodiments of the delivery detacher mechanism also have better visibility at the detacher which aids in melting a polymer for detachment. The improved visibility enhances ease of use. Embodiments of the delivery detacher mechanism have improved consistency of detachment.

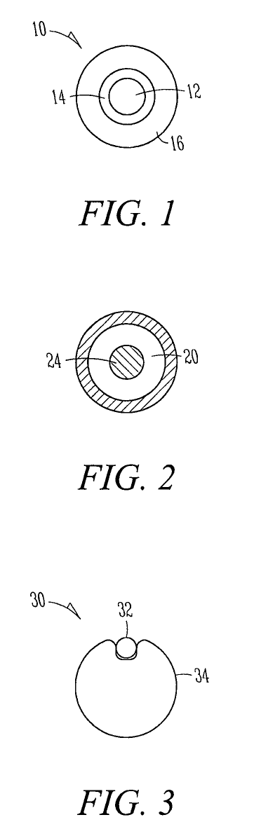

One embodiment of the proximal push support, illustrated at 10 in FIG. 1 includes a copper or silver core wire 12, an annular insulation layer 14 over the core wire and an annular layer 16 of Nitinol or other flexible metal over the insulation layer, as shown in FIG. 1. In another embodiment, shown at 20 in FIG. 2, includes a hypotube 22 is positioned over an insulated wire 24 as shown in FIG. 2. In another embodiment, shown at 30 in FIG. 3, the proximal push support includes an insulated core mechanism 32 and a Nitinol wire 34, wherein the insulated core wire 32 is positioned on an annular edge of the Nitinol wire.

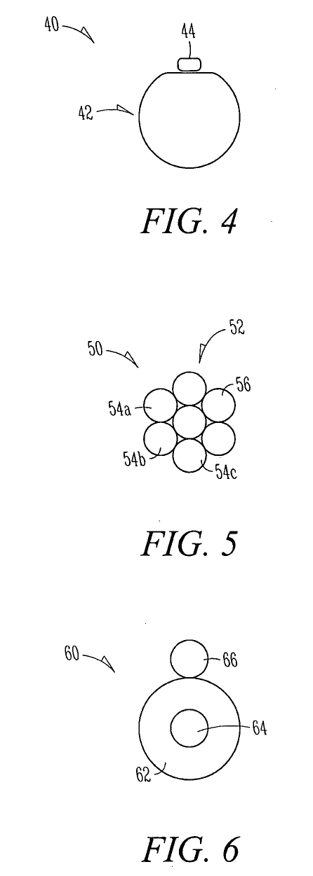

Another proximal support embodiment, shown at 40 in FIG. 4 includes a D-shaped wire 42 with an insulated flat wire 44. Another proximal support embodiment shown at 50 in FIG. 5, includes a cable 52 that includes multiple wires 54a, 54b, and 54c. One of the wires 56 is copper or silver insulated. Another proximal support embodiment, shown at 60 in FIG. 6, includes a Nitinol core wire 64, an annular coating 62 contacting the core wire 64, and an insulated copper wire 66, contacting the annular coating 62 as shown in FIG. 6.

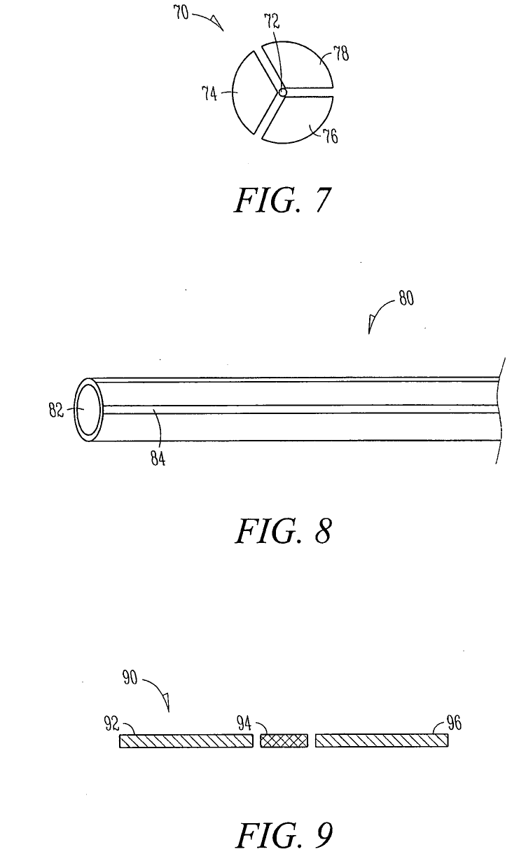

Another proximal support embodiment, shown at 70 in FIG. 7 includes an insulated copper wire 72 and pie-shaped wires 74, 76 and 78, arranged concentrically around the insulated copper wire 72.

Another proximal support embodiment, shown at 80 in FIG. 8, includes a Nitinol core wire 82, and a strip of conductive material, such as is shown at 84, extending along the length of the core wire 82.

One other proximal support embodiment, shown at 110 in FIG. 11, includes a Nitinol core wire 112, pie shaped wires 114, 116, 118, 120 and 122, enclosing the core wire 112, to form a core wire component 124. The core wire component 124 is positioned on another mechanism 126, as shown in FIG. 11.

One embodiment for increasing distal flexibility includes preparing a detacher mechanism that has a profile similar to the profile of the delivery mechanism.

One embodiment of a radiopaque tip/coil section includes sequenced coils, shown at 90 in FIG. 9. The sequenced coils 90 include an other coil 92, a heater coil 94 and a radiopaque coil 96. The sequenced coils 90 are insulated.

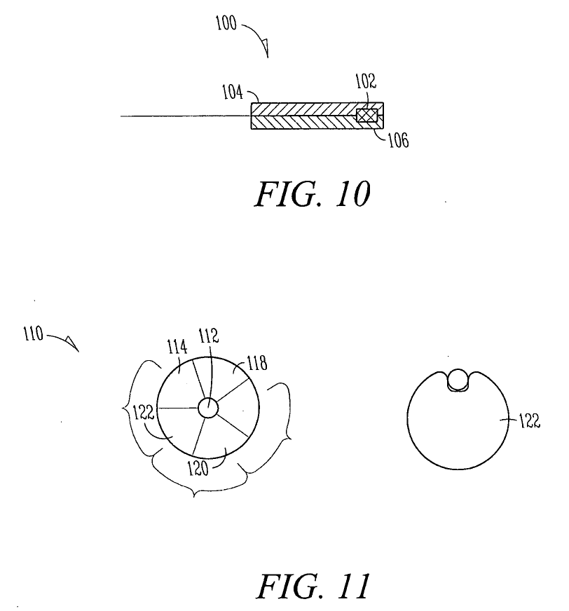

Another embodiment of a radiopaque tip/coil section, shown at 100 in FIG. 10, includes a heater coil 102 coated with an insulation coating 104. A radiopaque coating 106 is positioned under the insulation coating 104. In another embodiment, an insulated radiopaque coil is positioned under the heater coil.

For some embodiments, bismuth or bismuth alloys or both are used to impart or improve radiopacity. In one embodiment, a core mechanism or other mechanism is coated with a coating containing bismuth or bismuth alloys or both. With this bismuth coating, it is believed that a mechanism is visible and may be appropriately aligned with a heater coil. The mechanism maintains a high resistivity and conducts electricity and generates a desired heat zone to detach a polymer.

The aneurysm filler material described herein may be one or more of polymeric and polymeric hybrids such as PEBAX, Grilamids, polyester, and silica. Materials also include reabsorbables such as PGLA, PEG, PGLA and base polymer. Materials further include textiles such as rayon, nylon, silk, Kyeon, Kevlar, and cotton. Materials also include biopolymers such as collagen, filaments, and coated polymeric material. Materials further include elastomers such as urethanes, silicones, nitrites, Teco Flux, carbothane, and silicone hybrids

The textile materials may be knits or woven and may be expandable. The textiles include polybutester such as Novatyil, PGA (Dexon), PLA (polylactic acid), polyglactin acid (Vicryl), polydiaxanone (POS) and polylyconate (Maxon).

Pusher materials for the proximal shaft include Grilamids, nylon (12 30% glass (PARG)), polyamide, filled HDPE, polybutylene terephthalate, rigid polyurethane and polypropylene, that is 30% glass filled.

The aneurysm filler detacher mechanism embodiments may be formed by one or more processes such as reflow, thermal welding, adhesive welding, extrusion processing or other mechanisms for attaching two or more materials together.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

What is claimed is:

1. An aneurysm filler detacher mechanism comprising a proximal push support comprising a copper or silver core wire, an insulation layer positioned around the core wire, and an annular flexible layer positioned around the insulation layer.

2. The aneurysm filler detacher mechanism of claim 1 wherein the annular flexible layer comprises Nitinol.

3. An aneurysm filler detacher mechanism comprising a proximal push support comprising a core wire defining a channel and an insulated wire positioned within the channel.

4. An aneurysm filler detacher mechanism comprising a proximal push support comprising a core wire having a D-shape or a crescent shape and an insulted mechanism positioned on a flattened portion of the D-shape or crescent shape.

5. An aneurysm filler detacher mechanism comprising a proximal push support comprising a cable comprising a plurality of wires, wherein one of the wires is insulated.

6. An aneurysm filler detacher mechanism comprising a proximal push support comprising a DFT core wire and an insulated copper wire contacting the DFT wire.

7. An aneurysm filler detacher mechanism comprising a proximal push support comprising an insulated copper core wire and a plurality of pie-shaped wires positioned annularly about the core wire.

8. An aneurysm filler detacher mechanism comprising a proximal push support comprising an insulated core wire and a strip of conductive material extending along the core wire.

9. An aneurysm filler detacher mechanism comprising a radiopaque tip/coil section comprising a radiopaque coil, a heater coil and another coil, wherein the heater coil is positioned between the radiopaque coil and the other coil.

10. An aneurysm filler detacher mechanism comprising a radiopaque tip/coil section comprising: core wire coated with an insulation coating and a heater coil, and a radiopaque coating distal to the heater coil.

11. An aneurysm filler detacher mechanism comprising a mechanism coated with bismuth or bismuth alloy or both.
